(19) United States
(12) Patent Application Publication
(10) Pub. No.: US 2011/0158929 A1
(43) Pub. Date: Jun. 30, 2011

(54) METHOD FOR PRODUCING CROSS-LINKED ACRYLIC ACID POLYMERS

(75) Inventors: Son Nguyen Kim, Hemsbach (DE); Wolfgang Jahnel, Bellheim (DE)

(73) Assignee: BASF SE, Ludwigshafen (DE)

(21) Appl. No.: 12/674,581

(22) PCT Filed: Aug. 5, 2008
(86) PCT No.: PCT/EP2008/060258

§ 371 Date: Feb. 22, 2010

(30) Foreign Application Priority Data
Aug. 21, 2007 (EP) 07114682.3

(51) Int. Cl. A61K 8/81; C08F 2/38; A61Q 5/06; A61Q 5/12

(52) U.S. Cl. 424/70.16; 526/73

(57) ABSTRACT

The present invention relates to a method of producing crosslinked acrylic acid polymers, wherein the method is a precipitation polymerization which is carried out in a solvent comprising hydrocarbon and/or alkyl ester at least two different temperatures and with at least two different initiators. The present invention further relates to crosslinked polymers which are obtainable by the method according to the invention, and also to cosmetic and pharmaceutical preparations which comprise such polymers.

The present invention relates to a method of producing crosslinked acrylic acid polymers, wherein the method is a precipitation polymerization which is carried out in a solvent comprising hydrocarbon and/or alkyl ester at least two different temperatures and with at least two different initiators. The present invention further relates to crosslinked polymers which are obtainable by the method according to the invention, and also to cosmetic and pharmaceutical preparations which comprise such polymers.

Pulverulent compositions for rheology modification are known and are used in many fields such as surface coating, paper, textiles, hygiene, cosmetics or pharmaceuticals.

In the formulation of cosmetic or pharmaceutical preparations, such as, for example, skin creams and lotions, gels and other cosmetic care products and also pharmaceutical skin preparations, use is made, for the stabilization, of crosslinked acrylic acid polymers which are known under the CTFA name Carbomer. These polymers are precipitation polymers and are free-flowing powders which are neutralized by stirring them into water. This neutralization step is necessary in order to convert the acidic polymers to the carboxylates, which are ultimately responsible for the viscosity increase and thus stabilization of the formulation.

EP 328725 describes a method of producing a polymer by polymerizing a monomer mixture comprising at least 85% by weight of the monomer mixture of at least one oleofinically unsaturated carboxylic acid having 3 to 5 carbon atoms, salts of this (these) acid(s) and mixtures thereof, up to 15% by weight of this monomer mixture of at least one copolymerizable comonomer in the presence of a mixed reaction medium, up to 5% by weight of a crosslinking agent and less than 2.0% by weight of an initiator. The reaction medium comprises at least one organic solvent and at least one hydrocarbon solvent wherein the organic solvent is selected from ketones, esters, ethers and mixtures thereof and wherein the hydrocarbon solvent is selected from straight-chain aliphatics, branched-chain aliphatics, cyclic aliphatics and mixtures thereof, where the relative weight ratio of the at least one organic solvent to the at least one hydrocarbon solvent is in the range from 2:1 to 1:2.

DE 3750220 describes a method which is carried out in the presence of less than 3% water for producing a polymer of carboxylic monomer which comprises less than 0.2% of unreacted monomer, where more than 2% and up to 10% of the carboxyl groups of the monomer are neutralized, comprising the polymerization of a monomer charge which comprises at least 90% of an olefinically unsaturated carboxylic acid monomer having 3 to 5 carbon atoms and its salt, in the presence of a solvent selected from the group consisting of acetone, alkyl acetates having 1 to 6 carbon atoms in the alkyl group and mixtures thereof, in the presence of from 0.2 to 2.0% by weight of a monomer charge of a crosslinking agent and in the presence of less than 2% by weight of a monomer charge of an initiator selected from the group consisting of peroxydicarbonates, where the reaction temperature is from 45° C. to 55° C.

EP 371421 describes a method of producing acrylic acid copolymers where the acrylic acid monomer is polymerized in a nonaqueous solvent selected from acetone and an alkyl acetate having 1 to 6 carbon atoms in the alkyl group and in the presence of an initiator and divinyl glycol crosslinker, where the polymer is obtained in particulate form with an average particle size of less than 10 micrometers without grinding, giving rise to a slime viscosity of more than 50 000 mPas (cps) when measurement is made at 1% strength concentration in water.

WO 80/01164 describes a method of producing acrylic acid polymers where the acrylic acid is neutralized before the polymerization to a fraction of at least 1 mol % and the polymerization is carried out as precipitation polymerization in solvents with solubility parameters of from 8 to 15.

Disadvantages of the methods from the prior art are that the reaction times are sometimes very long, the solids contents of the polymerization mixtures are low and the amounts of residual monomers are high.

It was an object of the present invention to provide an improved method compared with the prior art which permits access to pulverulent thickener polymers, but at the same time does not have the disadvantages of the known methods.

This object was achieved by a method of producing crosslinked polymers by radically initiated precipitation polymerization of a monomer mixture comprising anionic and/or anionogenic monomers and less than 2% by weight of cationic and/or cationogenic monomers,
a. in the presence of a solvent which is or comprises at least one solvent selected from the group consisting of hydrocarbons, alkyl esters and mixtures thereof,
b. in the presence of at least two different polymerization initiators, where at least one of the initiators is a peroxide compound and
c. the temperature of the reaction mixture during the polymerization is increased at least once by at least 10° C.

WO 2007/010034 describes a method of producing ampholytic copolymers by radical precipitation polymerization using two different initiators. The polymerization of monomer mixtures which comprise less than 2% by weight of cationic and/or cationogenic monomers is not described.

In this connection, “during the polymerization” means the period of time which extends from the time point at which the polymerization starts to the time point at which at least 70% by weight, preferably at least 80% by weight, further preferably at least 90% by weight, of the monomers used have been polymerized.

“Monomer mixture” is understood as meaning the total amount of all radically polymerizable compounds which are used in the method according to the invention.

### Solvents

The method according to the invention is a precipitation polymerization. For this polymerization, solvents are used in which the starting materials for the polymerization are soluble and the resulting polymer is insoluble. The solvent used for the precipitation polymerization of the method according to the invention is or comprises at least one solvent selected from the group consisting of hydrocarbons, alkyl esters and mixtures thereof.

Preferred hydrocarbons are straight-chain, branched or cyclic hydrocarbons having 4 to 12 carbon atoms, further preferably those having 5 to 9 carbon atoms. Particular preference is given to n-pentane, n-hexane, n-heptane, n-octane, cyclohexane, cycloheptane, and aromatic hydrocarbons, such as benzene, toluene and xylene. The most preferred hydrocarbon constituent of the solvent is cyclohexane.

Preferred alkyl esters are esters of saturated aliphatic C1- to C4-carboxylic acids with monohydric saturated C1- to C6-alcohols. Further preference is given to the esters of acetic acid with monohydric saturated C1- to C6-alcohols, particular preference being given to methyl acetate, ethyl acetate, n-propyl acetate, isopropyl acetate, n-butyl acetate and sec-butyl acetate. Ethyl acetate, isopropyl acetate, n-butyl acetate and mixtures thereof are most preferred.

Preference is given to a method in which the solvent simultaneously comprises
a. at least one hydrocarbon, such as, for example, cyclohexane and
b. at least one alkyl ester, such as, for example, ethyl acetate.

### Polymerization Initiators

The method according to the invention is carried out in the presence of at least two different polymerization initiators A and B, where at least one of the initiators is a peroxide compound.

The method according to the invention is preferably one in which the two different polymerization initiators are insoluble in water. For the purposes of the present invention, insoluble in water means that less than 10 g, preferably less than 1 g, of the initiator dissolve in 1 liter of water under standard conditions to form a solution which is clear to the human eye.

The method according to the invention is preferably one in which the two different polymerization initiators are selected so that their particular decomposition temperatures are at least 45° C.

In the method according to the invention, preference is given to using at least two such polymerization initiators which permit essentially independent initiation in at least two phases. As a result, polymers with particularly low residual monomer contents are obtained.

For the polymerization, preference is given to using at least two polymerization initiators whose decomposition temperatures differ from one another by at least 10° C.

For the purposes of the invention, the decomposition temperature is defined as the temperature at which 50% of the molecules decompose into free radicals within 1 hour, i.e. the half-life time is 1 hour.

In one embodiment of the invention, the polymerization in this procedure takes place until precipitation of the polymer is complete at a temperature greater than or equal to the lower decomposition temperature and lower than the higher decomposition temperature, and, after the precipitation, a further reaction takes place at a temperature greater than or equal to the higher decomposition temperature.

In a preferred embodiment, the method according to the invention comprises a first polymerization phase at a first polymerization temperature and a second polymerization phase at a second polymerization temperature of at least 10° C. above the first polymerization temperature, where, for the polymerization, at least two initiators are used whose half-life times at the first polymerization temperature differ such that at least one of these initiators decomposes into radicals during the first polymerization phase and at least one of these initiators essentially does not decompose into radicals during the first polymerization phase and decomposes into radicals during the second polymerization phase.

Preferably, in this procedure, the second polymerization phase starts essentially after precipitation of the polymer. “Essentially” after precipitation of the polymer is understood as meaning that the polymer is present in precipitated form preferably to at least 70% by weight, preferably at least 80% by weight, in particular at least 90% by weight, based on the total weight of the polymer.

The half-life time of an initiator can be determined by customary methods known to the person skilled in the art, as described, for example, in the publication “Initiators for high polymers”, Akzo Nobel, No. 10737.

Preferably, the half-life time of the first polymerization initiator at the first polymerization temperature and of the second polymerization initiator at the second polymerization temperature is in a range from about 1 minute to 3 hours, particularly preferably 5 minutes to 2.5 hours. If desired, shorter half-life times, for example from 1 second to 1 minute, or half-life times longer than 3 hours, can also be used provided it is ensured that the initiator(s) decomposing at the higher temperature essentially does not decompose into radicals during the first polymerization phase but during the second polymerization phase.

In addition to the first and second polymerization phase, further polymerization phases can be used at different polymerization temperatures. Thus, for example, it is possible to carry out a first polymerization phase at a first polymerization temperature which is chosen such that controlled polymerization (i.e., for example, avoiding an undesired temperature increase as a result of the heat of reaction, an excessively high reaction rate, etc.) takes place. Subsequently, for example, an afterpolymerization can follow at a temperature which is above the first polymerization temperature and below the second polymerization temperature and which is chosen so that the initiator(s) decomposing at the higher temperature essentially does not decompose into radicals. Following completion of this afterpolymerization, to which, if desired, the initiator decomposing at the lower temperature and/or another initiator decomposing under the conditions of the afterpolymerization can again be added, the second polymerization phase can then follow.

In one embodiment of the invention, the initiator decomposing at the lower temperature has a decomposition temperature of from 50 to 100° C.

In one embodiment of the invention, the initiator decomposing at the higher temperature has a decomposition temperature of from 80 to 150° C.

In particular, the method is one in which the decomposition temperatures of the at least two polymerization initiators differ by at least 10° C., preferably by at least 14° C., further preferably by at least 17° C. and in particular by at least 20° C.

In one embodiment of the invention, the initiator decomposing at the higher temperature is initially introduced at the start of the copolymerization, or is added before or during the precipitation of the copolymer.

The water-insoluble initiators are preferably selected from water-insoluble diazo and peroxide compounds.

Polymerization initiators suitable for the method according to the invention are, for example, selected from dibenzoyl peroxide, succinyl peroxide, di-tert-butyl peroxide, tert-butyl perbenzoate, tert-butyl perpivalate, tert-butyl peroctoate, tert-butyl peroxy-2-ethylhexanoate, tert-butyl permaleate, benzoyl peroxide, tert-amyl peroxypivalate, cumene hydroperoxide, diisopropyl peroxydicarbamate, bis(o-toluoyl)peroxide, didecanoyl peroxide, dioctanoyl peroxide, dilauroyl peroxide, tert-butyl perisobutyrate, tert-butyl peracetate, di-tert-amyl peroxide, tert-butyl hydroperoxide, azobisisobutyronitrile, dimethyl 2,2′-azobis(2-methylpropionate) or 2,2′-azobis(2-methylbutyronitrile). Preferably, the polymerization initiators are selected from the group consisting of 2,5-dimethyl-2,5-di(tert-butylperoxy)hexane, di(2-ethylhexyl)peroxydicarbonate, tert-butyl peroxy-2-ethylhexanoate, benzoyl peroxide, tert-amyl peroxypivalate, dimethyl 2,2′-azobis(2-methylpropionate), tert-butyl peroxyneodecanoate, 2,2′-azobis(2-methylbutyronitrile), 2,2′-azobis(2-methylpropionamide), 2,2′-azobis(2,4-dimethylvaleronitrile). Furthermore, suitable initiators are 2,2′-azobis(4-methoxy-2,4-dimethylvaleronitrile), 1,1′-azobis(cyclohexane-1-carbonitrile), 2,2′-azobis[N-(2-propenyl)-2-methylpropionamide], 1-[(cyano-1-methylethyl)azo]formamide, 2,2′-azobis(N-butyl-2-methylpropionamide), 2,2′-azobis(N-cyclohexyl-2-methylpropionamide). The above-mentioned initiators are commercially available, for example, as Wako® and Trigonox® brands.

In one embodiment of the invention, it is preferred, for the polymerization up to a time point at which at least 70% by weight, preferably at least 80% by weight, particularly preferably at least 90% by weight and in particular at least 95% by weight, of the monomers used have been polymerized, to choose, as first initiator, that initiator whose decomposition temperature is at least 10° C. lower than that of the second initiator.

The second initiator is used in a preferred embodiment for the so-called afterpolymerization. The afterpolymerization is carried out after at least 80% by weight, preferably at least 90% by weight, particularly preferably at least 95% by weight and in particular at least 99% by weight, of the monomers used have been polymerized.

### Crosslinkers

Preferably, crosslinkers are used in an amount of from 0.01 to 2% by weight, particularly preferably 0.1 to 1% by weight, based on the total weight of the monomers used for the polymerization, including the crosslinkers.

Suitable crosslinkers are, for example, acrylic esters, methacrylic esters, allyl ethers or vinyl ethers of at least dihydric alcohols. The OH groups of the parent alcohols may here be completely or partially etherified or esterified; however, the crosslinkers comprise at least two ethylenically unsaturated groups.

Examples of the parent alcohols are dihydric alcohols, such as 1,2-ethanediol, 1,2-propanediol, 1,3-propanediol, 1,2-butanediol, 1,3-butanediol, 2,3-butanediol, 1,4-butanediol, but-2-ene-1,4-diol, 1,2-pentanediol, 1,5-pentanediol, 1,2-hexanediol, 1,6-hexanediol, 1,10-decanediol, 1,2-dodecanediol, 1,12-dodecanediol, neopentyl glycol, 3-methylpentane-1,5-diol, 2,5-dimethyl-1,3-hexanediol, 2,2,4-dimethyl-1,3-pentanediol, 1,2-cyclohexanediol, 1,4-cyclohexanediol, 1,4-bis(hydroxymethyl)cyclohexane, hydroxypivalic acid neopentyl glycol monoester, 2,2-bis(4-hydroxyphenyl)propane, 2,2-bis[4-(2-hydroxypropyl)phenyl]propane, diethylene glycol, triethylene glycol, tetraethylene glycol, dipropylene glycol, tripropylene glycol, tetrapropylene glycol, 3-thiopentane-1,5-diol, and polyethylene glycols, polypropylene glycols and polytetrahydrofurans with molecular weights of in each case 200 to 10 000. Apart from the homopolymers of ethylene oxide and propylene oxide, it is also possible to use block copolymers of ethylene oxide or propylene oxide or copolymers which comprise ethylene oxide and propylene oxide groups in incorporated form. Examples of parent alcohols having more than two OH groups are trimethylolpropane, glycerol, pentaerythritol, 1,2,5-pentanetriol, 1,2,6-hexanetriol, triethoxycyanuric acid, sorbitan, sugars, such as sucrose, glucose, mannose. The polyhydric alcohols can of course also be used following reaction with ethylene oxide or propylene oxide as the corresponding ethoxylates or propoxylates, respectively. The polyhydric alcohols can also firstly be converted to the corresponding glycidyl ethers by reaction with epichlorohydrin.

Further suitable crosslinkers are the vinyl esters or the esters of monohydric, unsaturated alcohols with ethylenically unsaturated C₃-C₆-carboxylic acids, for example acrylic acid, methacrylic acid, itaconic acid, maleic acid or fumaric acid. Examples of such alcohols are allyl alcohol, 1-buten-3-ol, 5-hexen-1-ol, 1-octen-3-ol, 9-decen-1-ol, dicyclopentenyl alcohol, 10-undecen-1-ol, cinnamyl alcohol, citronellol, crotyl alcohol or cis-9-octadecen-1-ol. However, it is also possible to esterify the monohydric, unsaturated alcohols with polybasic carboxylic acids, for example malonic acid, tartaric acid, trimellitic acid, phthalic acid, terephthalic acid, citric acid or succinic acid.

Further suitable crosslinkers are esters of unsaturated carboxylic acids with the above described polyhydric alcohols, for example of oleic acid, crotonic acid, cinnamic acid or 10-undecenoic acid.

Further suitable crosslinkers are urethane diacrylates and urethane polyacrylates, as are commercially available, for example, under the name Laromer®.

Also suitable as crosslinkers are straight-chain or branched, linear or cyclic, aliphatic or aromatic hydrocarbons which have at least two double bonds which, in the case of aliphatic hydrocarbons, must not be conjugated, e.g. divinylbenzene, divinyltoluene, 1,7-octadiene, 1,9-decadiene, 4-vinyl-1-cyclohexene, trivinylcyclohexane or polybutadienes with molecular weights of from 200 to 20 000.

Further suitable crosslinkers are the acrylamides, methacrylamides and N-allylamines of at least difunctional amines. Such amines are, for example, 1,2-diaminomethane, 1,2-diaminoethane, 1,3-diaminopropane, 1,4-diaminobutane, 1,6-diaminohexane, 1,12-dodecanediamine, piperazine, diethylenetriamine or isophoronediamine. Likewise suitable are the amides of allylamine and unsaturated carboxylic acids, such as acrylic acid, methacrylic acid, itaconic acid, maleic acid, or at least dibasic carboxylic acids, as have been described above.

Triallylamine and triallylmonoalkylammonium salts, e.g. triallylmethylammonium chloride or methyl sulfate, are also suitable as crosslinkers.

Also suitable are N-vinyl compounds of urea derivatives, at least difunctional amides, cyanurates or urethanes, for example of urea, ethyleneurea, propyleneurea or tartardiamide, e.g. N,N′-divinylethyleneurea or N,N′-divinylpropyleneurea.

Further suitable crosslinkers are divinyldioxane, tetraallylsilane or tetravinylsilane.

Mixtures of the abovementioned compounds can of course also be used. Particularly preferably used crosslinkers are, for example, methylenebisacrylamide, triallylamine and triallylalkylammonium salts, divinylimidazole, pentaerythritol triallyl ethers, N,N′-divinylethyleneurea, reaction products of polyhydric alcohols with acrylic acid or methacrylic acid, methacrylic acid esters and acrylic acid esters of polyalkylene oxides or polyhydric alcohols which have been reacted with ethylene oxide and/or propylene oxide and/or epichlorohydrin.

Very particularly preferred crosslinkers are pentaerythritol Wallyl ether, methylenebisacrylamide, N,N′-divinylethyleneurea, allyl (meth)acrylate, diallyltartardiamide, triallylamine and triallylmonoalkylammonium salts and acrylic acid esters of glycol, butanediol, trimethylolpropane or glycerol or acrylic acid esters of glycol, butanediol, trimethylolpropane or glycerol reacted with ethylene oxide and/or epichlorohydrin.

### Cationic and Cationogenic Monomers

For the purposes of this invention, “cationogenic” is understood as meaning a compound which can be converted to a cationic form by protonation or quaternization, in particular alkylation.

The monomer mixture to be polymerized comprises less than 4% by weight, preferably less than 2% by weight, particularly preferably less than 1% by weight and in particular less than 0.1% by weight, of cationic and/or cationogenic monomers. In one embodiment of the invention, the monomer mixture to be polymerized comprises no cationic and/or cationogenic monomers.

### Anionic and Anionogenic Monomers

The polymers obtainable according to the method of the invention comprise compounds with a radically polymerizable, α,β-ethylenically unsaturated double bond and at least one anionogenic and/or anionic group per molecule in copolymerized form. For the purposes of this invention, “anionogenic” is understood as meaning a compound which can be converted to an anionic form by deprotonation.

Preferably, these compounds comprise at least one compound which is selected from monoethylenically unsaturated carboxylic acids, sulfonic acids, phosphonic acids and mixtures thereof. The monomers include monoethylenically unsaturated mono- and dicarboxylic acids having 3 to 25, preferably 3 to 6, carbon atoms, which can also be used in the form of their salts or anhydrides. Examples thereof are acrylic acid, methacrylic acid, ethacrylic acid, α-chloroacrylic acid, crotonic acid, maleic acid, maleic anhydride, itaconic acid, citraconic acid, mesaconic acid, glutaconic acid, aconitic acid and fumaric acid. They further include the half-esters of monoethylenically unsaturated dicarboxylic acids having 4 to 10, preferably 4 to 6, carbon atoms, e.g. of maleic acid, such as monomethyl maleate. Furthermore, they also include monoethylenically unsaturated sulfonic acids and phosphonic acids, for example vinylsulfonic acid, allylsulfonic acid, sulfoethyl acrylate, sulfoethyl methacrylate, sulfopropyl acrylate, sulfopropyl methacrylate, 2-hydroxy-3-acryloxypropylsulfonic acid, 2-hydroxy-3-methacryloxypropylsulfonic acid, styrenesulfonic acid, 2-acrylamido-2-methylpropanesulfonic acid, vinylphosphonic acid and allylphosphonic acid. Furthermore, these also include the salts of the abovementioned acids, in particular the sodium, potassium and ammonium salts, and also the salts with amines. The anionic or anionogenic monomers can be used as such or as mixtures with one another.

Preferably, the anionic or anionogenic compound is or comprises at least one cornpound which is selected from acrylic acid, methacrylic acid, ethacrylic acid, α-chloroacrylic acid, crotonic acid, maleic acid, maleic anhydride, fumaric acid, itaconic acid, citraconic acid, mesaconic acid, glutaconic acid, aconitic acid, 2-acrylamido-2-methylpropanesulfonic acid, vinylphosphonic acid and mixtures thereof.

The anionic or anionogenic compound particularly preferably is or comprises at least one compound selected from acrylic acid, methacrylic acid and mixtures thereof. Most preferably, the anionic or anionogenic compound is or comprises acrylic acid.

The abovementioned anionic or anionogenic compounds can be used individually or in the form of any desired mixtures.

### Further Monomers

The polymers that can be produced by the method according to the invention can comprise further copolymerized monomers different from the anionic and/or anionogenic monomers. In general, all monomers radically copolymerizable with the anionic and/or anionogenic monomers are contemplated for this. Suitable monomers are given, for example, in WO 2007/010035, p. 33, I. 20 to p. 42, I. 22, to which reference is hereby made in its entirety.

Furthermore, preference is given to a method according to the invention in which the monomer mixture further comprises at least one monomer which is selected from C₅-C₃₀-(meth)acrylates, C₈-C₃₀-(meth)acrylamides, polyether (meth)acrylates terminated with C₈-C₃₀-alkyl groups and mixtures thereof, in particular with stearyl methacrylate, polyethylene glycol (meth)acrylates terminated with C₁₈-C₂₂-alkyl groups and mixtures thereof.

Suitable C₈-C₃₀-(meth)acrylates are, for example, pentyl(meth)acrylate, 2-pentyl(meth)acrylate, 3-pentyl(meth)acrylate, isopentyl acrylate, neopentyl acrylate, n-octyl(meth)acrylate, 1,1,3,3-tetramethylbutyl(meth)acrylate, ethylhexyl(meth)acrylate, n-nonyl(meth)acrylate, n-decyl(meth)acrylate, n-undecyl(meth)acrylate, tridecyl(meth)acrylate, myristyl(meth)acrylate, pentadecyl(meth)acrylate, palmityl(meth)acrylate, heptadecyl(meth)acrylate, nonadecyl(meth)acrylate, arrachinyl(meth)acrylate, behenyl(meth)acrylate, lignocerenyl(meth)acrylate, cerotinyl(meth)acrylate, melissinyl(meth)acrylate, palmitoleinyl(meth)acrylate, oleyl(meth)acrylate, linolyl(meth)acrylate, linolenyl(meth)acrylate, stearyl(meth)acrylate, lauryl(meth)acrylate, phenoxyethyl acrylate, t-butyl cyclohexylacrylate, cyclohexyl(meth)acrylate, ureido(meth)acrylate, tetrahydrofurfuryl(meth)acrylate and mixtures thereof.

Suitable C₅-C₃₀-(meth)acrylamides are, for example, N-(n-pentyl)(meth)acrylamide, N-(n-hexyl)(meth)acrylamide, N-(n-heptyl)(meth)acrylamide, N-(noctyl)(meth)acrylamide, N-(tert-octyl)(meth)acrylamide, N-(1,1,3,3-tetramethylbutyl)(meth)acrylamide, N-ethylhexyl(meth)acrylamide, N-(n-nonyl)(meth)acrylamide, N-(n-decyl)(meth)acrylamide, N-(n-undecyl)(meth)acrylamide, N-tridecyl(meth)acrylamide, N-myristyl(meth)acrylamide, N-pentadecyl(meth)acrylamide, N-palmityl(meth)acrylamide, N-heptadecyl(meth)acrylamide, N-nonadecyl(meth)acrylamide, N-arrachinyl(meth)acrylamide, N-behenyl(meth)acrylamide, N-lignocerenyl(meth)acrylamide, N-cerotinyl(meth)acrylamide, N-melissinyl(meth)acrylamide, N-palmitoleinyl(meth)acrylamide, N-oleyl(meth)acrylamide, N-linolyl(meth)acrylamide, N-linolenyl(meth)acrylamide, N-stearyl(meth)acrylamide, N-lauryl(meth)acrylamide and mixtures thereof.

A preferred method is one in which the monomer mixture comprises
i) 90 to 99.98% by weight of acrylic acid,
ii) 0 to 10% by weight of further monomers,
iii) 0.02 to 2% by weight of crosslinkers,
   with the proviso that the sum i) to iii) is 100% by weight.

### Afterpolymerization

The afterpolymerization is preferably carried out at a temperature greater than the temperature at which the main polymerization has been carried out.

In one embodiment, the temperature at which the afterpolymerization is carried out is at least 5° C., preferably at least 10° C., particularly preferably at least 15° C. and in particular at least 20° C., higher than the temperature of the main polymerization.

The amount of the initiator preferably used for the main polymerization is preferably from 0.001 to 2.0% by weight, further preferably 0.01 to 1.5% by weight, particularly preferably 0.05 to 1.0% by weight and in particular 0.1 to 0.5% by weight, based on the total amount of the monomers used.

The amount of the at least one initiator preferably used for the afterpolymerization is preferably from 0.001 to 2.5% by weight, further preferably 0.01 to 2.0% by weight, particularly preferably 0.1 to 1.5% by weight and in particular from 0.3 to 1.0% by weight, based on the total amount of the monomers used.

In a preferred embodiment of the invention, the amount by weight of the initiator preferably used for the afterpolymerization is greater than the amount by weight of the initiator preferably used for the main polymerization. It is particularly preferred if the amount by weight of the initiator preferably used for the afterpolymerization is at least 1.5 times, further preferably at least 2 times, particularly preferably at least 3 times, the amount by weight of the initiator preferably used for the main polymerization.

The main polymerization (first reaction phase) takes place in the temperature range from 40 to 110° C., preferably in the range from 50 to 100° C., particularly preferably in the range from 55 to 85° C. It is usually carried out under atmospheric pressure, but can also proceed under reduced or increased pressure, preferably from 1 to 5 bar.

The afterpolymerization (second or higher reaction phase) takes place in the temperature range from 50 to 200° C., preferably in the range from 70 to 160° C., particularly preferably in the range from 75 to 130° C. It is usually carried out under atmospheric pressure, but can also proceed under reduced or increased pressure, preferably from 1 to 5 bar.

The solvent or solvent mixture determines, through the corresponding boiling temperatures, the maximum reaction temperature if polymerization is carried out under atmospheric pressure. However, polymerizations under pressure are likewise possible.

Monomers and initiator are generally metered in over a period of from 1 to 10 hours, preferably from 2 to 5 hours.

During the production of the polymers, other polymers may, if appropriate, also be present, such as, for example, polyamides, polyurethanes, polyesters, homopolymers and copolymers of ethylenically unsaturated monomers. Examples of such polymers, sometimes also used in cosmetics, are the polymers known under the trade names Amerhold®, Ultrahold®, Ultrahold®Strong, Luviflex®VBM, Luvimer®, Acronal®, Acudyne®, Steppanhold®, Lovocryl®, Versatyl®, Amphomer® or Eastman AQ®, Luviset® brands, Sokalan® brands, Luviquat® brands.

Following the afterpolymerization step, the precipitated polymer is isolated from the reaction mixture, for which it is possible to use any customary method of isolating polymers in conventional precipitation polymerization. Such methods are filtration, centrifugation, evaporation of the solvent or combinations of these methods. To further purify the polymer of nonpolymerized constituents, the polymer may be washed. For this, it is possible in principle to use the same solvents as are suitable for the polymerization. On account of the advantageous method according to the invention, the amount of residual monomers is very small compared to known methods.

If the polymer is to be dried, then it is advisable to carry out a solvent exchange after the polymerization and to use low-boiling solvents for the drying.

### Interface-Active Substance

In a preferred embodiment of the invention, the method is one in which the precipitation polymerization is carried out in the presence of at least one interface-active substance. Preferably, this substance has an HLB value of less than or equal to 10.

Using the HLB-value (according to W. C. Griffin, J. Soc. Cosmetic Chem. 1 (1949) 311), emulsifiers can be classified according to the ratio of hydrophilic groups to lipophilic groups (HLB=hydrophilic-lipophilic balance).

Suitable interface-active substances with an HLB value of less than or equal to 10 are described, for example, in Karl-Heinz Schrader, Grundlagen and Rezepturen der Kosmetika [Fundamentals and Formulations of Cosmetics], 2nd edition, Verlag Hünig, Heidelberg, pp. 395-397, to which reference is made here in its entirety.

The determination of the HLB value of emulsifiers is known to the person skilled in the art and is described, for example, on p. 394 of the abovementioned literature reference. Further suitable interface-active substances with an HLB value of less than or equal to 10 are listed, for example, in U.S. Pat. No. 4,375,533, column 7, II. 26-60, to which reference is made here in its entirety. The use of interface-active substances in the precipitation polymerization of crosslinked polyacrylic acid is already described in U.S. Pat. No. 4,420,596 and U.S. Pat. No. 4,375,533.

The invention thus also provides a method as described above where the precipitation polymerization is carried out in the presence of at least one interface-active substance of linear block copolymers with a hydrophobic structural unit with a length of more than 5 nm (calculated by the law of cosines), which are defined by the following formula:

C_{w}—(B-A-B_{y})ₓ-D_{z}

in which
A is a hydrophilic structural unit which has a solubility in water at 25° C. of 1% by weight or more, has a molar mass M_{w} of from 200 to 50 000, and is selected such that it is bonded covalently to B;
B is a hydrophobic structural unit which has a molar mass M_{w} of from 300 to 60 000, has a solubility in water at 25° C. of less than 1% by weight and can be covalently bonded to A;
C and D are end groups which may be A or B and the same group or different groups;
w is 0 or 1;
x is an integer greater than or equal to 1,
y is 0 or 1, and
z is 0 or 1.

The invention further provides a method as described above where the precipitation polymerization is carried out in the presence of at least one interface-active substance made of random comb copolymers which are defined by the following formula:

R₁—(Z)ₘ—(Q)ₙ—R₂

in which
R₁ and R₂ are end groups and may be identical or different from one another and are different from Z and Q,
Z is a hydrophobic structural unit which has a solubility in water of 25° C. of less than 1% by weight,
Q is a hydrophilic structural unit which has a solubility in water of 25° C. of more than 1% by weight,
and
m and n are integers greater than or equal to 1 and are selected such that the molar mass M_{w} is from 100 to 250 000.

Interface-active substances are particularly preferably 12-hydroxystearic acid block copolymers, further preferably 12-hydroxystearic acid block copolymers with polyethylene oxide. The 12-hydroxystearic acid block copolymers are particularly preferably ABA block copolymers.
1) Hypermer® B239: block copolymer of a polyhydroxy fatty acid (PFA) and polyethylene oxide (PEO) with Mw of about 3500;
2) Hypermer® B246: block copolymer of a polyhydroxy fatty acid (PFA) and polyethylene oxide (PEO) with M_{w} of about 7500.
3) Hypermer® B261: block copolymer of a polyhydroxy fatty acid (PFA) and polyethylene oxide (PEO) with M_{w} of about 9600.
4) Hypermer® 2234: nonionic polymeric surface-active compound;
5) Hypermer® LP6: polymeric fatty acid ester with M_{w} of about 4300.
6) Hypermer® IL2296: nonionic polymeric surface-active compound;
7) Hypermer® A-109 block copolymer of a fatty acid or of a long-chain alkylene radical with ethylene oxide.
8) Hypermer® A-409 block copolymer of a fatty acid or of a long-chain alkylene radical with ethylene oxide.
9) Pecosil® PS-100 dimethicone copolyol phosphate polymer with 5-12 mol of ethylene oxide per mole of the hydrophilic unit.
10) Pecosil® WDS-100 dimethicone copolyol phosphate polymer with 5-12 mol of propylene oxide per mole of the hydrophilic unit.

Reference may be made at this point to the disclosure of EP 584771 B1, p. 23, 11.2 to 37, to which reference is made here in its entirety. EP 584771 B1 describes the use of such interface-active substances in the production of polyacrylic acid.

Furthermore, preference is given to a method according to the invention in which the interface-active substance is selected from the group consisting of
copolymers of polydimethylsiloxanes and organic glycols,
substances with the INCI name dimethicone PEG-7 phosphate,
polyesters comprising polyethylene glycol,
polyoxyethylene-glycerol-fatty-acid esters,
polyamide waxes,
natural waxes and
mixtures thereof.

Commercially available copolymers of polydimethylsiloxanes and organic glycols suitable according to the invention that may be mentioned are, for example, substances with the INCI name PEG/PPG-25/25 dimethicone (e.g. Belsil®DMC 6031) and dimethicone copolyol acetate (e.g. Belsil®DMC 6032).

A commercially available dimethicone PEG-7 phosphate (INCI) suitable according to the invention that may be mentioned is, for example, Pecosil®PS-100.

Commercially available polyesters comprising polyethylene glycol suitable according to the invention that may be mentioned are, for example, block copolymers of the Hypermer®, brand, in particular the grades B239, B246, B261, 2234, LP6, A-109, A-409 (described in EP 584771 B1, p. 10, II. 25-42).

Commercially available polyoxyethylene-glycerol-fatty-acid esters suitable according to the invention that may be mentioned are, for example, polyglyceryl-2 dipolyhydroxystearates (INCI) such as Dehymuls®PGPH (Cognis).

Also suitable according to the invention are compounds with the INCI names PEG-7 Hydrogenated Castor Oil, such as Arlacel®989, Cremophor®WO7 (BASF) or Dehymuls®HRE 7 (Cognis), PEG-2 Hydrogenated Castor Oil such as Arlacel®582, sorbitan monooleate/propylglyceryl 4/3-ricinoleate, such as Arlacel®1689 (Croda), sorbitan stearate and sucrose cocoate such as, for example, Arlartone®2121 (Croda), sorbeth-20 beeswax such as, for example, Atlas®G-1726 (Croda).

A commercially available polyamide wax suitable according to the invention that may be mentioned is, for example, Kahl Wax 6635 (Kahl&Co).

Commercially available natural waxes suitable according to the invention that may be mentioned are, for example, mixtures of fatty acid esters, fatty acid and fatty alcohol, such as, for example, beeswax, berry wax, rice wax (Kahl&Co).

Suitable beeswaxes are in particular those with the CAS numbers 8006-40-4 (white) or 8012-89-3. Suitable beeswaxes bear the INCI (EU) names Cera Alba, synthetic beeswax, PEG-7 dimethicone beeswax. Particularly suitable beeswaxes are those with the INCI EU name Cera Alba.

Suitable berry waxes are, for example, those with the INCI name Rhus Verniciflua Peel Wax (Berry Wax 6290 (Kahl & Co) or Botaniwax®OT (Botanigenics, Inc)).

Suitable rice waxes are in particular those with CAS number 8016-60-2 or the INCI name Oryza Sativa (rice) bran wax. Such rice waxes are available commercially as Cerewax® (Chemyunion Quimica LTDA), ESP®Rice Bran Wax (Earth Supplied Products, LLC), Florabeads®RBW (Floratech Americas), Naturebead®R20 (Micro Powders, Inc. Personal Care Division), Oryza Soft®“COS” (Cosmetochem International Ltd.), ORYZA® Wax (Ichimaru Pharcos Company, Ltd.), Ricebran Wax SP 8000 (Strahl & Pitsch, Inc.), Rice Wax No. 1(Tri-K Industries), Rice Wax 2811 (Kahl).

The amount of interface-active substances present during the precipitation polymerization is in the range from 0.001 to 50% by weight, preferably from 0.01 to 20% by weight and particularly preferably in the range from 0.1 to 10% by weight, based on the total amount of 100% by weight of the polymerizable compounds i) to iii), as defined above.

On account of their thickening effect, the polymers obtainable by the method according to the invention can be used as the sole gel former in cosmetic preparations. Moreover, they are also suitable for use in combination with customary gel formers.

### Neutralization

### Neutralization of the Monomers

The anionic and/or anionogenic monomers can be provided for the polymerization in, preferably partially, neutralized form. The neutralization can be carried out, for example, with an anhydrous, in particular a tertiary amine, such as triethanolamine, methyldiethanolamine, N,N-dimethyltriethanolamine or with an anhydrous base, such as Na₂CO₃, K₂CO₃. The degree of neutralization is preferably less than 20 mol %. The polymerization with partially neutralized acrylic acid has already been proposed in numerous patents, such as U.S. Pat. No. 3,915,921, U.S. Pat. No. 4,066,583, U.S. Pat. No. 4,758,641 or U.S. Pat. No. 4,267,103.

### Neutralization of the Polymers

The polymers obtainable by the method according to the invention act as thickeners in aqueous solution following at least partial neutralization.

Alkali metal and ammonium carbonate or hydrogen carbonate are suitable for the neutralization.

The neutralization can also take place with
a mono-, di- or trialkanolamine having 2 to 5 carbon atoms in the alkanol radical, which is present, if appropriate, in etherified form, for example mono-, di- and triethanolamine, mono-, di- and tri-n-propanolamine, mono-, di- and triisopropanolamine, 2-amino-2-methylpropanol and di(2-methoxyethyl)amine,
an alkanediolamine having 2 to 5 carbon atoms, for example 2-amino-2-methylpropane-1,3-diol and 2-amino-2-ethylpropane-1,3-diol, or
a primary, secondary or tertiary alkylamine having in total 5 to 10 carbon atoms, for example N,N-diethylpropylamine or 3-diethylamino-1-propylamine,
with N,N,N′,N′-tetrakis(2-hydroxypropyl)ethylenediamine (Neutrol®TE).

Suitable alkali metal hydroxides for the neutralization are in particular sodium hydroxide or potassium hydroxide and ammonium hydroxide.

Good neutralization results are often achieved using 2-amino-2-methylpropanol, triethanolamine, 2-amino-2-ethylpropane-1,3-diol, N,N-dimethylaminoethanol or 3-diethylamino-1-propylamine.

A neutralization until a degree of neutralization in the range from 90 to 100 mol % is reached or until a pH in the range from 6.5 to 7.5 is reached is preferred for a gel.

This neutralization of the polymers is preferably carried out with hydroxylamines such as AMP (aminomethylpropanol), TEA (triethanolamine), methyldiethanolamine and/or an inorganic base, such as NaOH or KOH.

Of suitability for the neutralization of the polymers in the preparations and compositions according to the invention are, in particular, also silicone polymers comprising amino groups. Suitable silicone polymers comprising amino grouops are, for example, the silicone-aminopolyalkylene oxide block copolymers of WO 97/32917, the products Silsoft®A-843 (dimethicone bisamino hydroxypropyl copolyol) and Silsoft®A-858 (trimethylsilyl ammodimethicone copolymer) (both Witco). Furthermore, the neutralization polymers of EP-A 1035144 and in particular the silicone-containing neutralization polymers of claim 12 in EP-A 1035144 are also suitable.

The polymer obtainable by the method according to the invention can be used wherever conventional thickeners based on polyacrylic acid are used.

The polymers obtainable by the method according to the invention can be used as thickeners in aqueous preparations in the sectors of household, personal care, building industry, textiles, for paper coating slips, pigment printing pastes, aqueous colors, leather-treatment compositions, cosmetic formulations, pharmaceutical products and agrochemicals.

The invention further provides the use of the polymers obtainable by the method according to the invention in cosmetic and/or pharmaceutical preparations.

The invention further provides cosmetic and/or pharmaceutical preparations comprising polymers obtainable by the method according to the invention.

The invention further provides cosmetic or pharmaceutical preparations comprising
A) at least one polymer obtainable by the method according to the invention and
B) at least one cosmetically acceptable carrier.

The preparations according to the invention preferably have a cosmetically or pharmaceutically acceptable carrier B) which is chosen from
i) water,
ii) water-miscible organic solvents, preferably C₂-C₄-alkanols, in particular ethanol,
iii) oils, fats, waxes,
iv) esters of C₆-C₃₀-monocarboxylic acids with mono-, di- or trihydric alcohols that are different from iii),
v) saturated acyclic and cyclic hydrocarbons,
vi) fatty acids,
vii) fatty alcohols,
viii) propellant gases,
   and mixtures thereof.

The preparations according to the invention have, for example, an oil or fatty component B) which is selected from: hydrocarbons of low polarity, such as mineral oils; linear saturated hydrocarbons, preferably having more than 8 carbon atoms, such as tetradecane, hexadecane, octadecane etc.; cyclic hydrocarbons, such as decahydronaphthalene; branched hydrocarbons; animal and vegetable oils; waxes; wax esters; vaseline; esters, preferably esters of fatty acids, such as, for example, the esters of C₁-C₂₄-monoalcohols with C₁-C₂₂-monocarboxylic acids, such as isopropyl isostearate, n-propyl myristate, isopropyl myristate, n-propyl palmitate, isopropyl palmitate, hexacosanyl palmitate, octacosanyl palmitate, triacontanyl palmitate, dotriacontanyl palmitate, tetratriacontanyl palmitate, hexacosanyl stearate, octacosanyl stearate, triacontanyl stearate, dotriacontanyl stearate, tetratriacontanyl stearate; salicylates, such as C₁-C₁₀-salicylates, e.g. octyl salicylate; benzoate esters, such as C₁₀-C₁₅-alkyl benzoates, benzyl benzoate; other cosmetic esters, such as fatty acid triglycerides, propylene glycol monolaurate, polyethylene glycol monolaurate, C₁₀-C₁₅-alkyl lactates, etc. and mixtures thereof.

Suitable silicone oils B) are, for example, linear polydimethylsiloxanes, poly(methylphenylsiloxanes), cyclic siloxanes and mixtures thereof. The number-average molecular weight of the polydimethylsiloxanes and poly(methylphenylsiloxanes) is preferably in a range from about 1000 to 150 000 g/mol. Preferred cyclic siloxanes have 4- to 8-membered rings. Suitable cyclic siloxanes are commercially available, for example, under the name cyclomethicone.

Preferred oil or fat components B) are selected from paraffin and paraffin oils; vaseline; natural fats and oils, such as castor oil, soya oil, peanut oil, olive oil, sunflower oil, sesame oil, avocado oil, cocoa butter, almond oil, peach kernel oil, ricinus oil, cod-liver oil, pig grease, spermaceti, spermaceti oil, sperm oil, wheat germ oil, macadamia nut oil, evening primrose oil, jojoba oil; fatty alcohols, such as lauryl alcohol, myristyl alcohol, cetyl alcohol, stearyl alcohol, oleyl alcohol, cetyl alcohol; fatty acids, such as myristic acid, stearic acid, palmitic acid, oleic acid, linoleic acid, linolenic acid and saturated, unsaturated and substituted fatty acids different therefrom; waxes, such as beeswax, carnauba wax, candililla wax, spermaceti, and mixtures of the abovementioned oil or fat components.

Suitable cosmetically and pharmaceutically compatible oil and fat components B) are described in Karl-Heinz Schrader, Grundlagen and Rezepturen der Kosmetika [Fundamentals and formulations of cosmetics], 2^{nd} edition, Verlag Hünig, Heidelberg, pp. 319-355, to which reference is made here.

Advantageously, those oils, fats and/or waxes are selected which are described on page 28, line 39 to page 34, line 22 of WO 2006/106140. Reference is hereby made to the content of the specified passage in its entirety.

The content of further oils, fats and waxes is at most 50% by weight, preferably 30% by weight, further preferably at most 20% by weight, based on the total weight of the composition.

Suitable hydrophilic carriers B) are selected from water, mono-, di- or polyhydric alcohols having preferably 1 to 8 carbon atoms, such as ethanol, n-propanol, isopropanol, propylene glycol, glycerol, sorbitol, etc.

The cosmetic compositions according to the invention may be skin cosmetic, hair cosmetic, dermatological, hygiene or pharmaceutical compositions. On account of their film-forming properties, the polymers obtainable by the method according to the invention are suitable in particular as thickeners for hair and skin cosmetics.

Preferably, the preparations according to the invention are in the form of a gel, foam, ointment, cream, emulsion, suspension, lotion, milk or paste. If desired, liposomes or microspheres can also be used.

The cosmetically or pharmaceutically active preparations according to the invention can additionally comprise cosmetically and/or dermatologically active ingredients, as well as auxiliaries.

Preferably, the cosmetic preparations according to the invention comprise at least one polymer A) obtainable by the method according to the invention, at least one carrier B) as defined above and at least one constituent different therefrom, which is selected from cosmetically active ingredients, emulsifiers, surfactants, preservatives, perfume oils, further thickeners, hair polymers, hair and skin conditioners, graft polymers, water-soluble or dispersible silicone-containing polymers, photoprotective agents, bleaches, gel formers, care agents, colorants, tinting agents, tanning agents, dyes, pigments, consistency regulators, humectants, refatting agents, collagen, protein hydrolyzates, lipids, antioxidants, antifoams, antistats, emollients and softeners.

The cosmetic compositions according to the invention can be present as aqueous or aqueous-alcoholic solutions, O/W and W/O emulsions, hydrodispersion formulations, solids-stabilized formulations, stick formulations, PIT formulations, in the form of creams, foams, sprays (pump spray or aerosol), gels, gel sprays, lotions, oils, oil gels or mousse and accordingly can be formulated with customary further auxiliaries.

Particularly preferred cosmetic compositions for the purposes of the present invention are gels, shampoos, washing and bathing preparations and also haircare compositions. The invention accordingly also relates to preparations for the cleansing and/or care of the hair and of the skin.

In particular, the invention relates to haircare compositions selected from the group consisting of styling gels, shampoos, hair conditioners, hair balms, pomades, styling creams, styling lotions, styling gels, end fluids, hot-oil treatments.

Furthermore, the invention relates to cosmetic preparations which are selected from gels, gel creams, hydroformulations, stick formulations, cosmetic oils and oil gels, mascara, self-tanning compositions, facecare compositions, bodycare compositions, after-sun preparations.

Further cosmetic preparations according to the invention are skin cosmetic preparations, in particular those for skincare. These are present in particular as W/O or O/W skin creams, day and night creams, eye creams, face creams, antiwrinkle creams, mimic creams, moisturizing creams, bleach creams, vitamin creams, skin lotions, care lotions and moisturizing lotions.

Furthermore, the polymers obtainable by the method according to the invention are suitable for skin cosmetic preparations, face masks, cosmetic lotions and for use in decorative cosmetics, for example for concealing sticks, stage make-up, in mascara and eye shadows, lipsticks, kohl pencils, eyeliners, make-up, foundations, blushers and powders and eyebrow pencils.

Furthermore, the preparations according to the invention can be used in antiacne compositions, repellents, shaving compositions, hair removal compositions, personal hygiene compositions, footcare compositions, and also in babycare.

Further preferred preparations according to the invention are washing, showering and bathing preparations which comprise the polymers obtainable by the method according to the invention.

For the purposes of this invention, washing, showering and bathing preparations are understood as meaning soaps of liquid to gel-like consistency, such as transparent soaps, luxury soaps, deodorant soaps, cream soaps, baby soaps, skin protection soaps, abrasive soaps and syndets, pasty soaps, soft soaps and washing pastes, liquid washing, showering and bathing preparations, such as washing lotions, shower baths and gels, foam baths, oil baths and scrub preparations, shaving foams, lotions and creams.

Suitable further ingredients for these washing, showering and bathing preparations according to the invention are described below.

Besides the polymeric thickeners obtainable by the method according to the invention, the preparations comprise further cosmetically acceptable additives, such as, for example, emulsifiers and coemulsifiers, solvents, surfactants, oil bodies, preservatives, perfume oils, cosmetic care and active ingredients, such as AHA acids, fruit acids, ceramides, phytantriol, collagen, vitamins and provitamins, for example vitamin A, E and C, retinol, bisabolol, panthenol, natural and synthetic photoprotective agents, natural substances, opacifiers, solubility promoters, repellents, bleaches, colorants, tinting agents, tanning agents (e.g. dihydroxyacetone), micropigments, such as titanium oxide or zinc oxide, super fatting agents, pearlescent waxes, consistency regulators, further thickeners, solubilizers, complexing agents, fats, waxes, silicone compounds, hydrotropes, dyes, stabilizers, pH regulators, reflectors, proteins and protein hydrolyzates (e.g. wheat, almond or pea proteins), ceramide, protein hydrolyzates, salts, gel formers, consistency regulators, further silicones, humectants (e.g. 1,2-pentanediol), refitting agents, UV photoprotective filters and further customary additives. Furthermore, to establish the properties desired in each case, it is in particular also possible for further polymers to be present.

The cosmetic preparations according to the invention comprise the polymers obtainable by the method according to the invention in an amount of from 0.01 to 10% by weight, preferably 0.05 to 5% by weight, particularly preferably 0.1 to 1.5% by weight, based on the weight of the preparation.

In a preferred embodiment of the invention, the inventive shower gels, washing, showering and bathing preparations, and shampoos and haircare compositions furthermore comprise at least one surfactant.

In a further preferred embodiment of the invention, besides the polymers, the shampoos and haircare compositions according to the invention furthermore comprise at least one oil and/or fatty phase and a surfactant.

### Surfactants

Surfactants which can be used are anionic, cationic, nonionic and/or amphoteric surfactants.

Advantageous washing-active anionic surfactants for the purposes of the present invention are
acylamino acids and salts thereof, such as acyl glutamates, in particular sodium acyl glutamate
sarcosinates, for example myristoyl sarcosine, TEA lauroyl sarcosinate, sodium lauroyl sarcosinate and sodium cocoyl sarcosinate, sulfonic acids and salts thereof, such as
acyl isethionates, for example sodium or ammonium cocoyl isethionate
sulfosuccinates, for example dioctyl sodium sulfosuccinate, disodium laureth sulfosuccinate, disodium lauryl sulfosuccinate and disodium undecylenamido MEA sulfosuccinate, disodium PEG-5 lauryl citrate sulfosuccinate and derivatives,

alkyl ether sulfates, for example sodium, ammonium, magnesium, MIPA, TIPA laureth sulfates, sodium myreth sulfate and sodium C₁₂₋₁₃ pareth sulfate,
alkyl ether sulfonates, for example sodium C12-15 pareth-15 sulfonate
alkyl sulfates, for example sodium, ammonium and TEA lauryl sulfate.

Further advantageous anionic surfactants are
taurates, for example sodium lauroyl taurate and sodium methyl cocoyl taurate,
ether carboxylic acids, for example sodium laureth-13 carboxylate and sodium PEG-6 cocamide carboxylate, sodium PEG-7 olive oil carboxylate
phosphoric acid esters and salts, such as, for example, DEA oleth-10 phosphate and dilaureth-4 phosphate,
alkyl sulfonates, for example, sodium coconut monoglyceride sulfate, sodium C₁₂₋₁₄ olefinsulfonate, sodium lauryl sulfoacetate and magnesium PEG-3 cocamide sulfate,
acyl glutamates, such as di-TEA palmitoyl aspartate and sodium caprylic/capric glutamate,
acyl peptides, for example palmitoyl hydrolyzed milk protein, sodium cocoyl hydrolyzed soya protein and sodium/potassium cocoyl hydrolyzed collagen,
   and also carboxylic acids and derivatives, such as, for example, lauric acid, aluminum stearate, magnesium alkanolate and zinc undecylenate, ester carboxylic acids, for example, calcium stearoyl lactylate, laureth-6 citrate and sodium PEG-4 lauramide carboxylate
alkylarylsulfonates.

Advantageous washing-active cationic surfactants for the purposes of the present invention are quaternary surfactants. Quaternary surfactants comprise at least one N atom which is covalently bonded to 4 alkyl or aryl groups. For example, alkylbetaine, alkylamidopropylbetaine and alkylamidopropylhydroxysultaine are advantageous. Further advantageous cationic surfactants for the purposes of the present invention are also
alkylamines,
alkylimidazoles and
ethoxylated amines
   and in particular salts thereof.

Advantageous washing-active amphoteric surfactants for the purposes of the present invention are acyl/dialkylethylenediamines, for example sodium acyl amphoacetate, disodium acyl amphodipropionate, disodium alkyl amphodiacetate, sodium acyl amphohydroxypropylsulfonate, disodium acyl amphodiacetate, sodium acyl amphopropionate, and N-coconut fatty acid amidoethyl N-hydroxyethylglycinate sodium salts. Further advantageous amphoteric surfactants are N-alkylamino acids, for example aminopropylalkylglutamide, alkylaminopropionic acid, sodium alkylimidodipropionate and lauroamphocarboxyglycinate.

Advantageous washing-active nonionic surfactants for the purposes of the present invention are
alkanolamides, such as cocamides MEA/DEA/MIPA,
esters which are formed by esterification of carboxylic acids with ethylene oxide, glycerol, sorbitan or other alcohols,
ethers, for example ethoxylated alcohols, ethoxylated lanolin, ethoxylated polysiloxanes, propoxylated POE ethers, alkyl polyglycosides, such as lauryl glucoside, decyl glycoside and cocoglycoside, glycosides with an HLB value of at least 20 (e.g. Belsil®SPG 128V (Wacker)).

Further advantageous nonionic surfactants are alcohols and amine oxides, such as cocoamidopropylamine oxide.

Preferred anionic, amphoteric and nonionic shampoo surfactants are specified, for example, in “Kosmetik and Hygiene von Kopf bis Fuβ” [Cosmetics and Hygiene from Head to Toe], Ed. W. Umbach, 3^{rd} edition, Wiley-VCH, 2004, pp. 131-134, to which reference is made at this point in its entirety.

Among the alkyl ether sulfates, sodium alkyl ether sulfates based on di- or triethoxylated lauryl and myristyl alcohol in particular are preferred. They are clearly superior to the alkyl sulfates with regard to insensitivity toward water hardness, ability to be thickened, low-temperature solubility and, in particular, skin and mucosa compatibility. They can also be used as sole washing raw materials for shampoos. Lauryl ether sulfate has better foam properties than myristyl ether sulfate, but is inferior to this as regards mildness.

Alkyl ether carboxylates are often used in combination with alkyl ether sulfates and amphoteric surfactants in hair washing compositions.

Sulfosuccinic acid esters (sulfosuccinates) are mild and readily foaming surfactants, but, on account of their poor ability to be thickened, are preferably only used together with other anionic and amphoteric surfactants and, on account of their low hydrolysis stability, are preferably only used in neutral and well buffered products.

Amidopropylbetaines as sole washing raw materials are unimportant in practice since their foaming behavior and also their ability to be thickened are only moderate. On the other hand, these surfactants have excellent skin and eye mucosa compatibility. In combination with anionic surfactants, their mildness can be synergistically improved. Preference is given to the use of cocamidopropylbetaine.

Amphoacetates/amphodiacetates have, as amphoteric surfactants, very good skin and mucosa compatibility and can have a hair-conditioning effect and/or enhance the care effect of additives. Similarly to the betaines, they are used for the optimization of alkyl ether sulfate formulations. Sodium cocoamphoacetate and disodium cocoamphodiacetate are most preferred.

Alkyl polyglycosides are nonionic washing raw materials. They are mild, have good universal properties, but are weakly foaming. For this reason, they are preferably used in combinations with anionic surfactants.

Sorbitan esters are likewise types of nonionic washing raw materials. On account of their excellent mildness, they are preferably employed for use in baby shampoos. Being weak foamers, they are preferably used in combination with anionic surfactants. It is advantageous to select the washing-active surfactant or surfactants from the group of surfactants which have an HLB value of more than 25, those which have an HLB value of more than 35 being particularly advantageous.

According to the invention, it is advantageous if one or more of these surfactants is used in a concentration of from 1 to 30% by weight, preferably in a concentration of from 5 to 25% by weight and very particularly preferably in a concentration of from 10 to 20% by weight, in each case based on the total weight of the preparation.

### Polysorbates

As washing-active agents, polysorbates can also advantageously be incorporated into the preparations according to the invention.

Polysorbates advantageous for the purposes of the invention are, for example,
polyoxyethylene(20) sorbitan monolaurate (Tween®20, CAS No. 9005-64-5)
polyoxyethylene(4) sorbitan monolaurate (Tween®21, CAS No. 9005-64-5)
polyoxyethylene(4) sorbitan monostearate (Tween®61, CAS No. 9005-67-8)
polyoxyethylene(20) sorbitan tristearate (Tween®65, CAS No. 9005-71-4)
polyoxyethylene(20) sorbitan monooleate (Tween®80, CAS No. 9005-65-6)
polyoxyethylene(5) sorbitan monooleate (Tween®81, CAS No. 9005-65-5)
polyoxyethylene(20) sorbitan trioleate (Tween®85, CAS No. 9005-70-3).
Polyoxyethylene(20) sorbitan monopalmitate (Tween®40, CAS No. 9005-66-7) and
polyoxyethylene(20) sorbitan monostearate (Tween®60, CAS No. 9005-67-8) are particularly advantageous.

The polysorbates are advantageously used in a concentration of from 0.1 to 5% by weight and in particular in a concentration of from 1.5 to 2.5% by weight, based on the total weight of the preparation, individually or as a mixture of two or more polysorbates.

### Conditioners

The conditioners selected for the cosmetic preparations according to the invention are preferably those which are described on page 34, line 24 to page 37, line 10 of WO 2006/106140. Reference is hereby made to the content of the specified passage in its entirety.

### Rheology Modifiers

The polymers obtainable by the method according to the invention generally bring about an adequate, very good thickening effect.

However, it is of course possible to additionally use further thickeners in the preparations according to the invention. Thickeners suitable for gels, shampoos and haircare compositions are given in “Kosmetik and Hygiene von Kopf bis Fuβ” [Cosmetics and Hygiene from Head to Toe], Ed. W. Umbach, 3^{rd} edition, Wiley-VCH, 2004, pp. 235-236, to which reference is made at this point in its entirety.

Suitable further thickeners for the cosmetic preparations according to the invention are described, for example, also on page 37, line 12 to page 38, line 8 of WO 2006/106140. Reference is hereby made to the content of the specified passage in its entirety.

### Preservatives

The cosmetic preparations according to the invention can also comprise preservatives.

Compositions with high water contents have to be reliably protected against the buildup of germs. Suitable preservatives for the cosmetic compositions according to the invention are described, for example, on page 38, line 10 to page 39, line 18 of WO 2006/106140. Reference is hereby made to the content of the specified passage in its entirety.

Complexing agents: since the raw materials and also many cosmetic compositions themselves are produced primarily in steel apparatuses, the end products can comprise iron (ions) in trace amounts. In order to prevent these impurities adversely affecting the product quality through reactions with dyes and perfume oil constituents, complexing agents such as salts of ethylenediaminetetraacetic acid, of nitrilotriacetic acid, of iminodisuccinic acid or phosphates are added.

UV photoprotective filters: in order to stabilize the ingredients present in the compositions according to the invention, such as, for example, dyes and perfume oils, against changes due to UV light, UV photoprotective filters, such as, for example, benzophenone derivatives, can be incorporated. Suitable UV photoprotective filters for the cosmetic compositions according to the invention are described, for example, on page 39, line 20 to page 41, line 10 of WO 2006/106140. Reference is hereby made to the content of the specified passage in its entirety.

Antioxidants: a content of antioxidants in the compositions according to the invention is generally preferred. According to the invention, antioxidants which can be used are all antioxidants customary or suitable for cosmetic applications. Suitable antioxidants for the cosmetic compositions according to the invention are described, for example, on page 41, line 12 to page 42, line 33 of WO 2006/106140. Reference is hereby made to the content of the specified passage in its entirety.

Buffers: buffers ensure the pH stability of the compositions. Citrate, lactate and phosphate buffers are primarily used.

Solubility promoters: they are used in order to bring care oils or perfume oils clearly into solution and also to keep them clearly in solution at low temperature. The most common solubility promoters are ethoxylated nonionic surfactants, for example hydrogenated and ethoxylated ricinus oils.

Antimicrobial agents: furthermore, antimicrobial agents can also be used. These include, in general, all suitable preservatives with specific action against gram-positive bacteria, e.g. triclosan (2,4,4′-trichloro-2′-hydroxy diphenyl ether), chlorhexidine (1,1′-hexamethylenebis[5-(4-chlorophenyl)biguanide), and TTC (3,4,4′-trichlorocarbanilide).

Quaternary ammonium compounds are in principle likewise suitable and are preferably used for disinfectant soaps and washing lotions. Numerous fragrances also have antimicrobial properties. A large number of essential oils or their characteristic ingredients, such as, for example, clove oil (eugenol), mint oil (menthol) or thyme oil (thymol), also exhibit marked antimicrobial effectiveness.

The antibacterially effective substances are generally used in concentrations of from about 0.1 to 0.3% by weight.

Dispersants: if insoluble active ingredients, e.g. antidandruff active ingredients or silicone oils, are to be dispersed or kept permanently in suspension in the compositions according to the invention, dispersants and thickeners, such as, for example, magnesium-aluminum silicates, bentonites, fatty acyl derivatives, polyvinylpyrrolidone or hydrocolloids, e.g. xanthan gum or carbomers, have to be used.

According to the invention, preservatives are present in a total concentration of at most 2% by weight, preferably at most 1.5% by weight and particularly preferably at most 1% by weight, based on the total weight of the composition.

Apart from the abovementioned substances, the compositions can, if appropriate, comprise the additives customary in cosmetics, for example perfume, dyes, refatting agents, complexing and sequestering agents, pearlizing agents, plant extracts, vitamins, active ingredients, pigments which have a coloring effect, softening, moisturizing and/or humectant substances, or other customary constituents of a cosmetic or dermatological formulation, such as alcohols, polyols, polymers, organic acids for adjusting the pH, foam stabilizers, electrolytes, organic solvents or silicone derivatives. With regard to the specified further ingredients known to the person skilled in the art for the compositions, reference may be made to “Kosmetik and Hygiene von Kopf bis Full” [Cosmetics and Hygiene from Head to Toe], Ed. W. Umbach, 3^{rd} edition, Wiley-VCH, 2004, pp. 123-128, to which reference is made at this point in its entirety.

The compositions according to the invention, such as gels, shampoos and haircare compositions, comprise, if appropriate, ethoxylated oils selected from the group of ethoxylated glycerol fatty acid esters, particularly preferably PEG-10 olive oil glycerides, PEG-11 avocado oil glycerides, PEG-11 cocoa butter glycerides, PEG-13 sunflower oil glycerides, PEG-15 glycerylisostearate, PEG-9 coconut fatty acid glycerides, PEG-54 hydrogenated ricinus oil, PEG-7 hydrogenated ricinus oil, PEG-60 hydrogenated ricinus oil, jojoba oil ethoxylate (PEG-26 jojoba fatty acids, PEG-26 jojoba alcohol), glycereth-5 cocoate, PEG-9 coconut fatty acid glycerides, PEG-7 glyceryl cocoate, PEG-45 palm kernel oil glycerides, PEG-35 ricinus oil, olive oil PEG-7 ester, PEG-6 caprylic acid/capric acid glycerides, PEG-10 olive oil glycerides, PEG-13 sunflower oil glycerides, PEG-7 hydrogenated ricinus oil, hydrogenated palm kernel oil glyceride PEG-6 ester, PEG-20 corn oil glycerides, PEG-18 glyceryl oleate cocoate, PEG-40 hydrogenated ricinus oil, PEG-40 ricinus oil, PEG-60 hydrogenated ricinus oil, PEG-60 corn oil glycerides, PEG-54 hydrogenated ricinus oil, PEG-45 palm kernel oil glycerides, PEG-80 glyceryl cocoate, PEG-60 almond oil glycerides, PEG-60 evening primrose glycerides, PEG-200 hydrogenated glyceryl palmate, PEG-90 glyceryl isostearate.

Preferred ethoxylated oils are PEG-7 glyceryl cocoate, PEG-9 coconut glycerides, PEG-40 hydrogenated ricinus oil, PEG-200 hydrogenated glyceryl palmate. Ethoxylated glycerol fatty acid esters are used in aqueous cleaning formulations for various purposes. Glycerol fatty acid esters with a degree of ethoxylation of about 30-50 serve as solubility promoters for nonpolar substances such as perfume oils. Highly ethoxylated glycerol fatty acid esters are used as thickeners.

### Active Ingredients

Highly diverse active ingredients of varying solubility can be incorporated homogeneously into the compositions according to the invention. Advantageous active ingredients in the cosmetic compositions according to the invention are described, for example, on page 44, line 24 to page 49, line 39 of WO 2006/106140. Reference is hereby made to the content of the specified passage in its entirety.

### UV Photoprotective Agents

Preferred preparations according to the invention are UV photoprotective preparations, in particular in the form of creams, lotions and sprays. These comprise UV photoprotective agents for protecting the skin and/or the hair. Suitable UV photoprotective agents are described in detail in WO 2006/106114, p. 24, 1.4 to p. 27, 1.27, to which reference is hereby made in its entirety.

The compositions advantageously comprise substances which absorb UV radiation in the UVB region and substances which absorb UV radiation in the UVA region, the total amount of the filter substances being, for example, 0.1 to 30% by weight, preferably 0.5 to 20% by weight, in particular 1 to 15% by weight, based on the total weight of the compositions, in order to provide cosmetic compositions which protect the skin from the entire range of ultraviolet radiation.

The majority of the photoprotective agents in the cosmetic or dermatological compositions serving to protect the human epidermis consists of compounds which absorb UV light in the UV-B region. For example, the fraction of the UV-A absorbers to be used according to the invention is 10 to 90% by weight, preferably 20 to 50% by weight, based on the total amount of substances absorbing UV-B and UV-A.

### Pearlescent Waxes

Suitable pearlescent waxes for the cosmetic compositions according to the invention are described, for example, on page 50, line 1 to line 16 of WO 2006/106140. Reference is hereby made to the content of the specified passage in its entirety.

The compositions according to the invention can furthermore comprise glitter substances and/or other effect substances (e.g. color streaks).

### Emulsifiers

In a preferred embodiment of the invention, the cosmetic compositions according to the invention are in the form of emulsions. Such emulsions are prepared by known methods. Suitable emulsifiers for the emulsions according to the invention are described, for example, on page 50, line 18 to page 53, line 4 of WO 2006/106140. Reference is hereby made to the content of the specified passage in its entirety.

### Perfume Oils

If perfume oils are to be added to the cosmetic compositions according to the invention, then suitable perfume oils are described, for example, on page 53, line 10 to page 54, line 3 of WO 2006/106140. Reference is hereby made to the content of the specified passage in its entirety.

### Pigments

If appropriate, the cosmetic compositions according to the invention furthermore comprise pigments. The pigments are present in the product mostly in undissolved form and may be present in an amount of from 0.01 to 25% by weight, particularly preferably from 0.5 to 15% by weight. The preferred particle size is 0.01 to 200 μm, in particular 0.1 to 150 μm, particularly preferably 1 to 100 μm.

Suitable pigments for the compositions according to the invention are described, for example on page 54, line 5 to page 55, line 19 of WO 2006/106140. Reference is hereby made to the content of the specified passage in its entirety.

### Polymers

In a preferred embodiment, apart from the polymers obtainable by the method according to the invention, the cosmetic compositions according to the invention also comprise further polymers. Preferred further polymers are water-soluble or water-dispersible polymers.

Further polymers suitable for the compositions according to the invention are described, for example, on page 55, line 21 to page 63, line 2 of WO 2006/106140. Reference is hereby made to the content of the specified passage in its entirety.

In particular, mention may be made here of N-vinylpyrrolidone polymers, such as poly-N-vinylpyrrolidone (PVP) and copolymers of N-vinylpyrrolidone with vinyl acetate, dimethylaminoethyl methacrylate or N-vinylimidazole.

The polymers obtainable by the method according to the invention are particularly suitable for producing hair-setting compositions and hair gels. For producing hair gels for styling the hair, the combination with so-called styling polymers is preferred. Examples of such styling gels are given in EP 1435226 B1, paragraphs [0020] to [0050], to which reference is hereby made in its entirety.

These styling polymers are present in the preparations preferably in an amount of 0.1 to 20% by weight, further preferably from 0.5 to 15% by weight, based on the preparation.

### Shampoo Types

A preferred embodiment of the invention are hair shampoos comprising the polymers obtainable by the method according to the invention. Additional requirements are, if appropriate, placed on shampoos according to hair quality or scalp problem. The mode of action of the preferred shampoo types with the most important additional effects or most important special objectives is described below.

According to the invention, preference is given, for example, to shampoos for normal or rapidly greasing or damaged hair, antidandruff shampoos, baby shampoos and two-in-one shampoos (i.e. shampoo and conditioner in one).

Shampoos according to the invention for normal hair: hair washing should free hair and scalp from the skin sebum formed in sebaceous glands, the inorganic salts emerging from sweat glands with water, amino acids, urea and lactic acid, shed skin particles, environmental dirt, odors and, if appropriate, residues of hair cosmetic treatments. Normal hair means short to shoulder-length hair which is only slightly damaged. Accordingly, the fraction of conditioning auxiliaries should be optimized to this hair type. Shampoos according to the invention for rapidly greasing hair: increased sebum production by the sebaceous glands on the scalp leads just 1-2 days after hair washing to a straggly, unkempt hairstyle. Oil- and wax-like skin sebum constituents weigh down the hair and reduce the friction from hair to hair and thus reduce the hairstyle hold. The actual hair cosmetic problem in the case of rapidly greasing hair is thus the premature collapse of voluminous hairstyles. In order to avoid this, it is necessary to prevent the hair surface from becoming weighed down and too smooth and supple. This is preferably achieved through the surfactant base of highly cleaning washing raw materials that are characterized by particularly low substantivity. Additional care substances which would add to the skin sebum, such as refatting substances, are used in shampoos for rapidly greasing hair only with the greatest of care, if at all. Volumizing shampoos according to the invention for fine hair can be formulated comparably.

Shampoos according to the invention for dry, stripped (damaged) hair: the structure of the hair is changed in the course of hair growth by mechanical influences such as combing, brushing and primarily back-combing (combing against the direction of growth), by the effect of UV radiation and visible light and by cosmetic treatments, such as permanent waves, bleaching or coloring. The flake layer of the hair has an increasingly stripped appearance from root to the end; in extreme cases, it is completely worn away at the end, and the hair ends are split (split ends). Damaged hair can in principle no longer be returned to the state of healthy hair regrowth. However, it is possible to come very close to this ideal state as regards feel, shine and combability by using shampoos according to the invention with, if appropriate, high fractions of care substances (conditioners).

An even better hair conditioning effect than with a shampoo is achieved with a haircare composition according to the invention, for example in the form of a rinse or cure treatment after hair washing. Rinses or cures for hair which comprise polymers obtainable by the method according to the invention are likewise in accordance with the invention.

2-in-1 shampoos according to the invention are particularly high-care shampoos in which, as a result of the design as “shampoo and conditioner in one” the additional care benefit is placed equally alongside the basic cleaning benefit. 2-in-1 compositions according to the invention comprise increased amounts of conditioners.

Antidandruff shampoos: compared with antidandruff hair tonics, antidandruff shampoos according to the invention have the advantage that they not only reduce the formation of new visible flakes through appropriate active ingredients against dandruff attack and prevent such formation upon long-term application, but also remove flakes already shed with the hair washing. However, after rinsing out the wash liquor, only a small, but adequate amount of the active ingredients remains on the scalp and hair. There are various antidandruff active ingredients which can be incorporated into the shampoo compositions according to the invention, such as, for example, zinc pyrithion, ketoconazole, elubiol, clotrimazole, climbazole or piroctone olamine. Additionally, these substances have a normalizing effect on shedding.

The basis of antidandruff shampoos corresponds primarily to the formulation of shampoos for normal hair with a good cleaning effect.

Baby shampoos: in a preferred embodiment of the invention, the shampoo preparations according to the invention are baby shampoos. These are optimally skin- and mucosa-compatible. Combinations of washing raw materials with very good skin compatibility form the basis of these shampoos. Additional substances for further improving the skin and mucosa compatibility and the care properties are advantageously added, such as, for example, nonionic surfactants, protein hydrolyzates and panthenol or bisabolol. All of the required raw materials and auxiliaries, such as preservatives, perfume oils, dyes etc., are selected from the aspect of high compatibility and mildness. Shampoos for dry scalp: in a further preferred embodiment of the invention, the shampoo preparations according to the invention are shampoos for dry scalp. The primary aim of these shampoos is to prevent the scalp from drying out since dry scalp can lead to irritation, reddening and inflammation. As also in the case of the baby shampoos, combinations of washing raw materials with very good skin compatibility form the basis of these shampoos. Additionally, if appropriate, refatting agents and humectants, such as, for example, glycerol or urea, can be used.

The shampoo compositions according to the invention can also be present as shampoo concentrates with increased surfactant contents of 20-30% by weight. They are based on special washing raw material combinations and consistency regulators which ensure good spreadability and the spontaneous foaming ability even of a small application amount. A particular advantage is, for example, the possibility of achieving the productivity of 200 ml of shampoo with a 100 ml bottle.

### Supply Form

It is advantageous if the compositions according to the invention are stored in a bottle or squeezable bottle and are applied from this. Accordingly, bottles or squeezable bottles which comprise a composition according to the invention are also in accordance with the invention.

The polymers obtainable by the method according to the invention, as defined above, can preferably be used in shampoo formulations in particular as conditioners. Preferred shampoo formulations comprise
a) 0.05 to 10% by weight of at least one polymer obtainable by the method according to the invention,
b) 25 to 94.95% by weight of water,
c) 5 to 50% by weight of surfactants,
c) 0 to 5% by weight of a further conditioner,
d) 0 to 10% by weight of further cosmetic constituents.

All anionic, neutral, amphoteric or cationic surfactants customarily used in shampoos can be used in the shampoo formulations. Suitable surfactants have been specified above.

### Soaps and Syndets

Further compositions according to the invention which comprise the polymers obtainable by the method according to the invention are, for example, soaps and syndets. Soap is formed in the reaction of a (neutral) fat or fatty acids obtained therefrom or fatty acid methyl esters with sodium hydroxide or potassium hydroxide solution (saponification). Soap is chemically the alkali metal salt of fatty acids in the composition. The neutral fats usually used in the manufacture of soap are beef tallow or palm oil in a mixture with coconut oil or palm kernel oil and—more rarely—other natural oils or fats, the quality of the starting fats being highly influential on the quality of the soap obtained therefrom.

Of importance for selecting the fatty components is the distribution of the chain lengths of the corresponding fatty acids. Normally, especially C12-C18-fatty acids are in demand. Since laurate soap foams particularly well, lauric-rich coconut oil or similarly formulated palm kernel oil are usually used in relatively high fractions (up to 50% of the neutral fatty mixture) for soaps for which a large amount of foam during use is desired. The sodium salts of the specified fatty acid mixtures are solid, whereas the potassium salts are soft and pasty. For this reason, the hydroxide solution component used for producing solid soaps is preferably sodium hydroxide solution, and for liquid-pasty soaps is preferably potassium hydroxide solution. During the saponification, the ratio of hydroxide solution to fatty acid is selected so that, at most, a minimum excess of hydroxide solution (max. 0.05%) is present in the finished soap bar.

The soaps usually include toilet, curd, transparent, luxury, cream, freshening/deodorant, baby, skin protection, abrasive, floating and liquid soaps and also washing pastes and soap leaves.

Besides the polymers obtainable by the method according to the invention, soaps according to the invention advantageously furthermore comprise antioxidants, complexing agents and humectants, and, if appropriate, fragrances, dyes and further cosmetically acceptable ingredients. Such further suitable ingredients are specified above.

Syndets (synthetic detergents) are alternatives to conventional soaps which have certain advantages as a result of the varying composition compared to soap, whereas soap more likely has disadvantages.

Syndets comprise, as foam and cleaning components, washing-active substances (surfactants) which are obtained by chemical synthesis. By contrast, soaps are—as described—salts of naturally occurring fatty acids. For syndets, skin-mild, readily biodegradable surfactants are used, preferably fatty acid isethionates (sodium cocoyl isethionate), sulfosuccinic acid half-esters (disodium lauryl sulfosuccinate), alkyl polyglucosides (decyl glucoside), amphoteric surfactants (e.g. sodium cocoamphoacetate). In addition, monoglyceride sulfate and ether carboxylates sometimes play a role. Fatty alcohol sulfate (e.g. sodium lauryl sulfate) has largely lost its former significance as base surfactant for syndets. The base surfactants are combined with builder substances, refatting agents and further additives to give formulations which can be processed by customary soap technology and produce bars which behave as far as possible in a “soap-like” manner, but without the mentioned disadvantages of soap. They foam at every water hardness and have a very good cleaning power. Their pH can be adjusted within a wide range (mostly between 4 and 8).

On account of the more intensive cleaning/degreasing power of the base surfactants, the surfactant fraction in the syndet is usually significantly lower, the fraction of super fatting agents is significantly higher than in soaps, without the foaming ability being reduced. Syndets are recommended specifically for the cleansing of sensitive skin, of youthful-blemished skin and for face washing.

Alongside the (soap-free) syndets is also found the market segment of half- or combars (derived from combination bar). These are bars which comprise both soap and syndet surfactants. Combars comprise 10 to 80% by weight of soap. They represent a compromise between soaps and syndets for the criteria of costs, foaming ability, skin feel and compatibility. When washing with a combar, a pH of from about 7 to 9 is established, depending on its soap fraction.

As regards possible formulations for soaps and syndets known to the person skilled in the art, reference may be made to “Kosmetik und Hygiene von Kopf bis Full” [Cosmetics and Hygiene from Head to Toe], Ed. W. Umbach, 3^{rd} edition, Wiley-VCH, 2004, pp. 112-122, to which reference is made at this point in its entirety.

### Shower Bath and Bathing Products

As regards specific compositions for shower bath and bathing products or washing lotions, reference may be made to “Kosmetik und Hygiene von Kopf bis Full” [Cosmetics and Hygiene from Head to Toe], Ed. W. Umbach, 3^{rd} edition, Wiley-VCH, 2004, pp. 128-134, to which reference is made at this point in its entirety.

The invention further provides the use of a polymer obtainable by the method according to the invention as thickener in pharmacy for modifying rheological properties.

A preferred subject matter of the invention is cosmetic preparations in the form of a hair gel or a shampoo which comprise the polymers obtainable by the method according to the invention.

The polymers obtainable by the method according to the invention are particularly suitable as thickeners in hair gels, in particular so-called styling gels. A preparation suitable according to the invention for styling gels can, for example, have the composition as follows:
a) 60-99.85% by weight of water and/or alcohol
b) 0.05-10% by weight of a polymer obtainable by the method according to the invention
c) 0.1-20% by weight of a styling polymer
d) 0-20% by weight of further constituents

The polymers obtainable by the method according to the invention can be used as gel formers on their own or together with further, customary gel formers. Such further, customary gel formers are lightly crosslinked polyacrylic acid, for example Carbomer (INCI), cellulose derivatives, e.g. hydroxypropylcellulose, hydroxyethylcellulose, cationically modified celluloses, polysaccharides, e.g. xanthum gum, caprylic/capric triglycerides, sodium acrylates copolymer, Polyquaternium-32 (and) paraffinum liquidum (INCI), sodium acrylates copolymer (and) paraffinum liquidum (and) PPG-1 trideceth-6, acrylamidopropyltrimonium chloride/acrylamide copolymer, steareth-10 alkyl ether acrylates copolymer, Polyquaternium-37 (and) paraffinum liquidum (and) PPG-1 trideceth-6, Polyquaternium 37 (and) propylene glycol dicaprate dicaprylate (and) PPG-1 trideceth-6, Polyquaternium-7, Polyquaternium-44.

The polymers obtainable by the method according to the invention can be used very well as thickeners in shampoo formulations.

Preferred shampoo formulations comprise
a) 0.05-10% by weight of the polymer obtainable by the method according to the invention
b) 25-94.95% by weight of water
c) 5-50% by weight of surfactants
d) 0-5% by weight of a conditioner
e) 0-10% by weight of further cosmetic constituents

In the shampoo formulations it is possible to use all anionic, neutral, amphoteric or cationic surfactants customarily used in shampoos.

Suitable anionic surfactants are, for example, alkyl sulfates, alkylethersulfates, alkylsulfonates, alkylarylsulfonates, alkyl succinates, alkyl sulfosuccinates, N-alkoyl sarcosinates, acyl taurates, acyl isethionates, alkyl phosphates, alkyl ether phosphates, alkyl ether carboxylates, alpha-olefinsulfonates, in particular the alkali metal and alkaline earth metal salts, e.g. sodium, potassium, magnesium, calcium, and ammonium and triethanolamine salts. The alkyl ether sulfates, alkyl ether phosphates and alkyl ether carboxylates can have between 1 to 10 ethylene oxide or propylene oxide units, preferably 1 to 3, ethylene oxide units, in the molecule.

For example, sodium lauryl sulfate, ammonium lauryl sulfate, sodium lauryl ether sulfate, ammonium lauryl ether sulfate, sodium lauroyl sarcosinate, sodium oleyl succinate, ammonium lauryl sulfosuccinate, sodium dodecylbenzenesulfonate, triethanolamine dodecylbenzenesulfonate are suitable.

Suitable amphoteric surfactants are, for example, alkylbetaines, alkylamidopropylbetaines, alkylsulfobetaines, alkyl glycinates, alkylcarboxyglycinates, alkyl amphoacetates or -propionates, alkyl amphodiacetates or -dipropionates.

For example, cocodimethylsulfopropylbetaine, laurylbetaine, cocamidopropylbetaine or sodium cocamphopropionate can be used.

Suitable nonionic surfactants are, for example, the reaction products of aliphatic alcohols or alkylphenols having 6 to 20 carbon atoms in the alkyl chain, which may be linear or branched, with ethylene oxide and/or propylene oxide. The amount of alkylene oxide is about 6 to 60 mols per mole of alcohol. Furthermore, alkylamine oxides, mono- or dialkylalkanolamides, fatty acid esters of polyethylene glycols, alkyl polyglycosides or sorbitan ether esters are suitable.

Furthermore, the shampoo formulations can comprise customary cationic surfactants, such as, for example, quaternary ammonium compounds, for example cetyltrimethylammonium chloride.

In the shampoo formulations, customary conditioners can be used in combination with the polymers obtainable by the method according to the invention to achieve certain effects. These include, for example, cationic polymers with the INCI name Polyquaternium, in particular copolymers of vinylpyrrolidone/N-vinylimidazolium salts (Luviquat®FC, Luviquat®HM, Luviquat®MS, Luviquat®Care), copolymers of N-vinylpyrrolidone/dimethylaminoethyl methacrylate, quaternized with diethyl sulfate (Luviquat®PQ 11), copolymers of N-vinylcaprolactam/N-vinylpyrrolidone/N-vinylimidazolium salts (Luviquat®Hold); cationic cellulose derivatives (Polyquaternium-4 and -10), acrylamide copolymers (Polyquaternium-7). It is also possible to use protein hydrolyzates, and also conditioning substances based on silicone compounds, for example polyalkylsiloxanes, polyarylsiloxanes, polyarylalkylsiloxanes, polyether siloxanes or silicone resins. Further suitable silicone compounds are dimethicone copolyols (CTFA) and amino functional silicone compounds, such as amodimethicones (CTFA).

Preferred preparations which comprise the polymer obtainable by the method according to the invention are:
shampoos
hairstyling gels
hydroalcoholic gels
moisturizing gels
bath gels
hand, body and face lotions
creams
sunscreen lotions

### EXAMPLES

### Meaning of the Abbreviations/Trade Names

AA acrylic acid
SMA stearyl methacrylate
PETAE pentaerythritol triallyl ether
Perpivalate tert-butyl perpivalate
Plex-6877 O® 25% strength solution of C₁₈-alkyl PEG₁₁₀₀ methacrylate in methyl methacrylate (Degussa)
AMA allyl methacrylate (Röhm)
Wako®V59 2,2′-azobis(2-methylbutyronitrile)
Wako®V65 2,2′-azobis(2,4-dimethylvaleronitrile)
Trigonox®23 tert-butyl peroxyneodecanoate
Trigonox®101 2,5-dimethyl-2,5-di(tert-butylperoxy)hexane
Trigonox®EHP di(2-ethylhexyl)peroxydicarbonate
TBPOC tert-butyl peroctoate
Hypermer®B 246 polyethylene glycol-polyester block copolymer (Uniqema)
Dehymuls®PGPH polyglyceryl-2 dipolyhydroxystearate (Cognis)
Wax Rice Wax 2811 (Kahl)

### Example S2

### Method with an Initiator

| | | | |
|---|---|---|---|
| Initial charge: | cyclohexane | 370 | g |
| | butyl acetate | 370 | g |
| | tert-butyl peroctoate | 0.25 | g |
| | feed 1 | 250 | g |
| Feed 1 | cyclohexane | 180 | g |
| | butyl acetate | 180 | g |
| | acrylic acid | 500 | g |
| | pentaerythritol triallyl ether | 2.5 | g |
| Feed 2 | cyclohexane | 140 | g |
| | butyl acetate | 140 | g |
| | tert-butyl peroctoate | 2.5 | g |
| Feed 3 | cyclohexane | 400 | g |
| | butyl acetate | 400 | g |

The initial charge was heated to about 60° C. while passing nitrogen through and stirring. Feed 1 was metered in over the course of 4 hours. After about 15 min., the onset of clouding was observed. 30 minutes after starting feed 1, feed 2 was metered in over the course of 4.5 hours. 60 minutes after starting feed 1, feed 3 was metered in over the course of 4 hours. When feed 2 was completed, the mixture was heated to 70° C. and stirred for a further 3 hours. The mixture was then heated to 80° C. and stirred for a further 3 hours at this temperature. Then, the suspension was afterpolymerized at 90° C. for 2 hours. The product was isolated by filtration and dried at about 70° C. for 20 hours.

Polymerization was carried out analogously in Experiment S1 in ethyl acetate with Wako®V59 at a temperature of 80° C.

### Example 11

### Method (I), According to the Invention

| | | | |
|---|---|---|---|
| Initial charge: | cyclohexane | 370 | g |
| | ethyl acetate | 370 | g |
| | Wako ®V65 | 0.1 | g |
| | tert-butyl peroctoate | 1.25 | g |
| | feed 1 | 250 | g |
| Feed 1 | cyclohexane | 180 | g |
| | ethyl acetate | 180 | g |
| | acrylic acid | 500 | g |
| | pentaerythritol triallyl ether | 2.5 | g |
| Feed 2 | cyclohexane | 140 | g |
| | ethyl acetate | 140 | g |
| | Wako ®V65 | 0.15 | g |
| | tert-butyl peroctoate | 1.25 | g |
| Feed 3 | cyclohexane | 400 | g |
| | ethyl acetate | 400 | g |

The initial charge was heated to about 60° C. while passing nitrogen through and stirring. Feed 1 was metered in over the course of 4 hours. After about 15 min., the onset of clouding was observed. 30 minutes after starting feed 1, feed 2 was metered in over the course of 4.5 hours. 60 minutes after starting feed 1, feed 3 was metered in over the course of 4 hours. When feed 2 was completed, the mixture was heated to 7° C. and stirred for a further 3 hours. The mixture was then heated to 80° C. and stirred for a further 3 hours at this temperature. Then, the suspension was afterpolymerized at 90° C. for 2 hours. The product was isolated by filtration and dried at about 70° C. for 20 hours.

The experiments of Examples 1 to 13 were carried out analogously.

### Example 14

### Method (II), According to the Invention

| | | | |
|---|---|---|---|
| Initial charge: | cyclohexane | 380 | g |
| | ethyl acetate | 380 | g |
| | Dehymuls ®PGPH | 15 | g |
| | Wako ®V65 | 0.25 | g |
| | tert-butyl peroctoate | 2.5 | g |
| | feed 1 | 250 | g |
| Feed 1 | cyclohexane | 175 | g |
| | ethyl acetate | 175 | g |
| | acrylic acid | 500 | g |
| | pentaerythritol triallyl ether | 2.5 | g |
| Feed 2 | cyclohexane | 250 | g |
| | ethyl acetate | 250 | g |

The initial charge was heated to about 60° C. while passing nitrogen through and stirring. Feed 1 was metered in over the course of 4 hours. After about 15 min., the onset of clouding was observed. 30 minutes after starting feed 1, feed 2 was metered in over the course of 4.5 hours. When feed 2 was completed, the mixture was heated to 75° C. and stirred for a further 3 hours. The mixture was then heated to 90° C. and stirred for a further 3 hours at this temperature. Then, the suspension was afterpolymerized at 90° C. for 2 hours. The product was isolated by filtration and dried at about 70° C. for 20 hours.

The experiments of Examples 14 to 21 were carried out analogously.

### Example 23

### Method (III)

| | | | |
|---|---|---|---|
| Initial charge: | butyl acetate | 750 | g |
| | Hypermer ®B 246 | 15 | g |
| | Wako ®V59 | 0.25 | g |
| | Trigonox ®101 | 2.5 | g |
| | feed 1 | 280 | g |
| Feed 1 | butyl acetate | 350 | g |
| | acrylic acid | 500 | g |
| | pentaerythritol triallyl ether | 2.5 | g |
| Feed 2 | butyl acetate | 1100 | g |

The initial charge was heated to about 75° C. while passing nitrogen through and stirring. Feed 1 was metered in over the course of 4 hours. After about 15 min., the onset of clouding was observed. 30 minutes after starting feed 1, feed 2 was metered in over the course of 4.5 hours. When feed 2 was completed, the mixture was heated to 75° C. and stirred for a further 2 hours. The mixture was then heated to 8° C. and stirred for a further 3 hours at this temperature. Then, the suspension was afterpolymerized at 120° C. for 1 hour. The product was isolated by filtration and dried at about 70° C. for 20 hours.

Examples 22, 24 and 25 were carried out analogously in a pressurized apparatus.

| | | | | | Interface- | | Initiator A | Initiator B | AA: residual | |
|---|---|---|---|---|---|---|---|---|---|---|
| | | | Plex-O | Cross- | active | | (reaction | (reaction | monomer | |
| Example | AA | SMA | 6877 | linker | substance | Solvent | temperature) | temperature) | [ppm] | Method |
| S1 | 100 | — | — | PETAE | — | ethyl acetate | Wako ®V59 | Wako ®V59 | ca. 1500 | standard |
| | | | | 0.5 | | | (80° C.) | (80° C.) | | method |
| | | | | | | | | | | (an initiator) |
| S2 | 100 | — | — | PETAE | — | butyl | TBPOC | TBPOC | ca. 800 | an initiator |
| | | | | 0.5 | | acetate/ | (60° C.) | (95° C.) | | two temp. |
| | | | | | | cyclohexane | | | | |
| 1 | 100 | — | — | PETAE | — | cyclohexane | Trigonox ®23 | TBPOC | <50 | method (I) |
| | | | | 0.5 | | | (60° C.) | (70°-90° C.) | | |
| 2 | 100 | — | — | PETAE | — | cyclohexane | Wako ®V65 | TBPOC | <100 | method (I) |
| | | | | 0.4 | | | (60° C.) | (70°-90° C.) | | |
| 3 | 100 | — | — | PETAE | — | ethyl | Trigonox ®23 | TBPOC | <30 | method (I) |
| | | | | 0.5 | | acetate/ | (60° C.) | (70°-90° C.) | | |
| | | | | | | cyclohexane | | | | |
| 4 | 100 | — | — | PETAE | — | cyclohexane | Perpivalat | TBPOC | <100 | method (I) |
| | | | | 0.4 | | | (70° C.) | 85 | | |
| 5 | 100 | — | — | PETAE | — | cyclohexane | Wako ®V65 | TBPOC | <100 | method (I) |
| | | | | 0.4 | | | (60° C.) | (70°-90° C.) | | |
| 6 | 98 | 2 | — | PETAE | — | cyclohexane | Perpivalat | TBPOC | <120 | method (I) |
| | | | | 0.5 | | | (70° C.) | (70°-90° C.) | | |
| 7 | 100 | — | — | PETAE | — | isopropyl | Wako ®V65 | TBPOC | <30 | method (I) |
| | | | | 0.5 | | acetate | (60° C.) | (70°-90° C.) | | |
| 8 | 100 | — | — | AMA | — | isopropyl | Wako ®V65 | TBPOC | <50 | method (I) |
| | | | | 0.5 | | acetate | (60° C.) | (70°-90° C.) | | |
| 9 | 90 | — | 10 | PETAE | — | ethyl | Wako ®V65 | TBPOC | <30 | method (I) |
| | | | | 0.15 | | acetate/ | (60° C.) | (70°-90° C.) | | |
| | | | | | | cyclohexane | | | | |
| 10 | 90 | — | 10 | AMA | — | ethyl acetate | Trigonox ®EHP | TBPOC | <40 | method (I) |
| | | | | 0.2 | | | (60° C.) | (70°-90° C.) | | |
| 11 | 100 | — | — | PETAE | — | ethyl | Wako ®V65 | TBPOC | <30 | method (I) |
| | | | | 0.5 | | acetate/ | (60° C.) | (70°-90° C.) | | |
| | | | | | | cyclohexane | | | | |
| 12 | 97 | 3 | — | PETAE | — | ethyl | Wako ®V65 | TBPOC | <30 | method (I) |
| | | | | 0.4 | | acetate/ | (60° C.) | (70°-90° C.) | | |
| | | | | | | cyclohexane | | | | |
| 13 | 95 | — | 5 | PETAE | — | ethyl | Wako ®V65 | TBPOC | <30 | method (I) |
| | | | | 0.4 | | acetate/ | (60° C.) | (70°-90° C.) | | |
| | | | | | | cyclohexane | | | | |
| 14 | 100 | — | — | PETAE | Dehymuls 3 | ethyl | Wako ®V65 | TBPOC | <50 | method (II) |
| | | | | 0.5 | | acetate/ | (60° C.) | (70°-90° C.) | | |
| | | | | | | cyclohexane | | | | |
| 15 | 100 | — | — | AMA | Hypermer 3 | ethyl | Wako ®V65 | TBPOC | <20 | method (II) |
| | | | | 0.6 | | acetate/ | (60° C.) | (70°-90° C.) | | |
| | | | | | | cyclohexane | | | | |
| 16 | 100 | — | — | PETAE | Hypermer 2 | ethyl | Wako ®V65 | TBPOC | <20 | method (II) |
| | | | | 0.6 | | acetate/ | (60° C.) | (70°-90° C.) | | |
| | | | | | | cyclohexane | | | | |
| 17 | 100 | — | — | PETAE | Wax 2811 3 | ethyl | Wako ®V65 | TBPOC | <20 | method (II) |
| | | | | 0.5 | | acetate/ | (60° C.) | (70°-90° C.) | | |
| | | | | | | cyclohexane | | | | |
| 18 | 100 | — | — | PETAE | Hypermer + | ethyl | Wako ®V65 | TBPOC | <20 | method (II) |
| | | | | 0.5 | Wax 2811 | acetate/ | (60° C.) | (70°-90° C.) | | |
| | | | | | 2 + 2 | cyclohexane | | | | |
| 19 | 98 | 2 | — | PETAE | Hypermer 2 | ethyl | Wako ®V65 | TBPOC | <20 | method (II) |
| | | | | 0.5 | | acetate/ | (60° C.) | (70°-90° C.) | | |
| | | | | | | cyclohexane | | | | |
| 20 | 100 | — | — | PETAE | Dehymuls 3 | ethyl | Wako ®V65 | TBPOC | <20 | method (II) |
| | | | | 0.5 | | acetate/ | (60° C.) | (70°-90° C.) | | |
| | | | | | | cyclohexane | | | | |
| 21 | 100 | — | — | PETAE | Hypermer 5 | ethyl | Wako ®V65 | TBPOC | <20 | method (II) |
| | | | | 0.5 | | acetate/ | (60° C.) | (70°-90° C.) | | |
| | | | | | | cyclohexane | | | | |
| 22 | 100 | — | — | PETAE | Dehymuls 3 | ethyl | TBPOC | Trigonox ®101 | <20 | method (III) |
| | | | | 0.5 | | acetate/ | (70° C + 80° C.) | (120° C.) | | |
| | | | | | | cyclohexane | | | | |
| 23 | 100 | — | — | PETAE | — | butyl acetate | Wako ®V59 | Trigonox ®101 | <100 | method (III) |
| | | | | 0.5 | | | (80° C.) | (120° C.) | | |
| 24 | 98 | 2 | — | AMA | — | ethyl | Trigonox ®EHP | Trigonox ®101 | <100 | method (III) |
| | | | | 0.5 | | acetate/ | (60° C.) | (120° C.) | | |
| | | | | | | cyclohexane | | | | |
| 25 | 98 | 2 | — | PETAE | — | ethyl | Trigonox ®EHP | Trigonox ®101 | <100 | method (III) |
| | | | | 0.5 | | acetate/ | (60° + 80 C) | (120° C.) | | |
| | | | | | | cyclohexane | | | | |

1.-12. (canceled)

13. A method of producing crosslinked polymers by radically initiated precipitation polymerization of a monomer mixture comprising anionic and/or anionogenic monomers and less than 2% by weight of cationic and/or cationogenic monomers,
a. in the presence of a solvent which is or comprises at least one solvent selected from the group consisting of hydrocarbons, alkyl esters and mixtures thereof,
b. in the presence of at least two different polymerization initiators A and B, where at least one of the initiators is a peroxide compound and
c. the temperature of the reaction mixture during the polymerization is increased at least once by at least 10° C.

14. The method according to claim 13, wherein the anionic or anionogenic monomers are selected from the group consisting of acrylic acid, methacrylic acid, maleic acid, maleic anhydride, their salts and mixtures.

15. The method according to claim 13, wherein the solvent simultaneously comprises
a. at least one hydrocarbon and
b. at least one alkyl ester.

16. The method according to claim 13, wherein the monomer mixture further comprises at least one monomer which is selected from C₅-C₃₀-(meth)acrylates, C₅-C₃₀-(meth)acrylamides, polyether (meth)acrylates terminated with C₅-C₃₀-alkyl groups, and mixtures thereof.

17. The method according to claim 13, wherein the monomer mixture comprises
i) 90 to 99.98% by weight of acrylic acid,
ii) 0 to 10% by weight of further monomers,
iii) 0.02 to 2% by weight of crosslinkers,
with the proviso that the sum i) to iii) is 100% by weight.

18. The method according to claim 13, wherein the precipitation polymerization is carried out in the presence of at least one interface-active substance with an HLB value of less than or equal to 10.

19. The method according to claim 18, wherein the interface-active substance is selected from the group consisting of
copolymers of polydimethylsiloxanes and organic glycols,
substances with the INCI name dimethicone PEG-7 phosphate,
polyesters comprising polyethylene glycol,
polyoxyethylene-glycerol-fatty-acid esters,
polyamide waxes,
natural waxes and
mixtures thereof.

20. A crosslinked polymer obtainable by a method according to claim 13.

21. A cosmetic and/or pharmaceutical preparation comprising polymers obtainable by a method according to claim 13.

22. The cosmetic preparation according to claim 21 in the form of a shampoo, hairstyling gel, hydroalcoholic gel, moisturizing gel, bath gel, a hand, body or face lotion, cream or sunscreen lotion.

23. A styling gel which comprises the composition as follows:
a) 60-99.85% by weight of water and/or alcohol
b) 0.05-10% by weight of a polymer obtainable by the method according to claim 13,
c) 0.1-20% by weight of a styling polymer and
d) 0-20% by weight of further constituents.

24. A shampoo formulation which comprises
a) 0.05-10% by weight of the polymer obtainable by the method according to claim 13,
b) 25-94.95% by weight of water
c) 5-50% by weight of surfactants
d) 0-5% by weight of a conditioner and
e) 0-10% by weight of further cosmetic constituents.
